(19) United States
(12) Patent Application Publication
(10) Pub. No.: US 2022/0268730 A1
(43) Pub. Date: Aug. 25, 2022

(54) Relative Quantitation Using Electrochemical Mass Spectrometry

(71) Applicant: NEW JERSEY INSTITUTE OF TECHNOLOGY, Newark, NJ (US)

(72) Inventors: Hao Chen, Livingston, NJ (US); Pengyi Zhao, Harrison, NJ (US)

(73) Assignee: NEW JERSEY INSTITUTE OF TECHNOLOGY, Newark, NJ (US)

(21) Appl. No.: 17/678,295

(22) Filed: Feb. 23, 2022

Related U.S. Application Data
(60) Provisional application No. 63/152,927, filed on Feb. 24, 2021.

(51) Int. Cl. G01N 27/447; G01N 27/416; G01N 27/453

(52) U.S. Cl. CPC G01N 27/44721; G01N 27/4168; G01N 27/453

(57) ABSTRACT

A method for relative quantification of organic and biological compounds by electrochemical mass spectrometry is disclosed. The method involves using electrochemistry (EC) in a mass spectrometry (MS)-based relative quantitative analysis. In this method, isotope-labeled standards or running calibration curves are not employed. A quantification method could include the steps of subjecting a sample analyte to liquid chromatography or electrophoresis separation, followed by an electrochemical oxidation or reduction in an electrochemical cell, and then mass spectrometric detection.

### CROSS-REFERENCE TO RELATED APPLICATIONS

The present application claims the benefit under 35 U.S.C. § 119(e) to U.S. Provisional Appl. No. 63/152,927, filed Feb. 24, 2021, which is hereby incorporated by reference in its entirety.

### FIELD OF THE DISCLOSURE

The present disclosure relates to quantification of target compounds. In particular, the present disclosure relates to relative quantification of organic and biological compounds by mass spectrometry in combination with electrochemistry.

### BACKGROUND

Relative quantitation refers to measuring quantity of one sample relative to another sample. Relative quantitative mass spectrometry (MS) has been widely used in metabolomics, peptidomics, and proteomics research areas.¹ Relative quantitative MS is based on comparing the level of analyte of interest in a sample to the same analyte in a different sample. Generally, relative quantitation methods based on MS can be classified into two categories: label-free and isotope labeling methods. Each category is discussed below.

In the label-free approach, samples are separately measured by MS and the ion intensities of the analyte from different samples are directly compared. Higher ion intensity indicates higher abundance of the analyte in the sample. However, because the ion signal is affected by instrumental conditions in addition to analyte concentration, the label-free method is not highly accurate and suffers from the fluctuation of instrumental conditions.

Turning now to the isotope labeling method, the samples with an analyte that are light and heavy isotope-labeled can be merged and measured by MS in a single run. The relative ion intensity ratio of the light-isotope labeled versus the heavy-isotope labeled analytes can provide information about the relative quantity of the analyte in different samples. Methods using this approach include stable isotope labeling by amino acids in cell culture (SILAC),² tandem mass tags (TMT),^{3,4} isotope-coded affinity tags (ICAT),⁵ and isobaric tags for relative and absolute quantitation (iTRAQ),⁶ etc., Many of these methods such as ICAT, SILAC, and TMT have been made marketable by major instrument companies such as Thermo Scientific (San Jose, Calif.) and Sciex (Ontario, Calif.). However, isotope-labeled standard synthesis can be expensive, labor-intense, and time-consuming. For example, 1 mg of ¹³C or ¹⁵N labeled peptide costs several hundred dollars and takes about two to four weeks to prepare. In addition, they are not 100% pure and most synthetic peptides have a purity of only 95%. During storage, these isotope-labeled standards can also degrade with time.

In recent reported studies for the combination of electrochemistry (EC) and mass spectrometry (MS),⁷⁻⁹ a method was disclosed that can be applied to absolute quantitation of an analyte such as neurotransmitters, uric acid, oligosaccharide, and peptides. Absolute quantitation refers to determining the actual quantity of an analyte in each sample such as concentration or mass of the analyte in the sample. The results have been reported in three journal articles⁷⁻⁹ and one patent.¹⁰ In particular, International Publication No. WO 2018/081228 discloses a method for absolute quantification using MS.¹⁰

Electrochemistry (EC) can be combined with liquid chromatography (LC) where the electrochemical cell can serve as a detector for LC. In such LC/EC experiments, the proteins, peptides and glycans can be separated and detected by electrochemical detectors¹¹⁻¹². A DC potential or pulsed potential can be used for triggering oxidation of analytes.

There is an unmet need for new methods of quantitation that can overcome the shortcomings of the methods described above.

### SUMMARY

In accordance with embodiments of the present disclosure, methods and systems for relative quantification of organic and biological compounds by electrochemical mass spectrometry (EC/MS) are disclosed. In various embodiments, an electrochemical signal is used for relative quantitation of compounds (e.g., proteins) without the use of a standard, and a mass spectrometric signal is used for identification of the target compound(s). Such EC/MS methods can be further coupled with LC to build a LC/EC/MS platform where a real-world mixture sample can be subjected to separation first, followed by electrochemical conversion (for relative quantitation purposes) and then MS detection (for identification purposes).

In one or more embodiments, a method is disclosed using electrochemistry in a mass spectrometry-based relative quantitative analysis. This method can be applicable to a variety of samples, including small organic molecules, such as drugs, drug metabolites, and organic pollutants as well as large biological samples such as lipids, carbohydrates, peptides, proteins, and nucleic acids. In this method, isotope-labeled standards or running calibration curves are not employed. Thus, embodiments of the present method can be cost-effective and fast. The method can be used to quantify drug metabolites, protein and peptide drugs, degraded products of organic compounds, species for biological or biomedical research, etc.

In one embodiment, a quantification process could include the steps of subjecting a sample analyte to liquid chromatography (LC) or electrophoresis separation, followed by an electrochemical oxidation or reduction in an electrochemical cell, and then mass spectrometric detection. In another embodiment, a quantification system could include an electrochemical cell connected to a mass spectrometer. Liquid chromatography, electrophoresis or direct infusion could be employed in this embodiment.

In one embodiment, a method for quantifying a target compound in a sample is disclosed. The method could include the steps of applying an oxidation/reduction potential to an electrochemical cell containing the target compound, thereby causing a rise of an electrochemical current signal. The current signal is proportional to the concentration of the target compound. A controller could be included to measure the current signal of the target compound.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF DRAWINGS

To assist those of skill in the art in making and using the disclosed relative quantification system and method and associated systems and methods, reference is made to the accompanying figures, wherein:

FIG. 1 illustrates a process flow diagram of a method according to an embodiment of the present disclosure;

FIG. 2 is a schematic of an electrochemical cell according to an embodiment of the present disclosure;

FIG. 3 is a schematic of a relative quantification system, in accordance with one embodiment of the present disclosure;

FIG. 4A is a graphical depiction showing a linear relationship between electric charge Q and the concentration of peptide DRVY, a chosen analyte;

FIG. 4B is a graphical depiction showing ESI-MS spectra of peptide DRVY when the applied potential was a) 0 V and b) +1.05 V; the peak of the oxidation product of DRVY was seen at m/z 550;

FIG. 5A is a schematic showing sequence of insulin protein;

FIG. 5B is a plot of electric charge Q vs. different concentrations of insulin;

FIG. 6A is a schematic illustration of an antibody structure; and

FIG. 6B is a plot of electric charge Q vs. different concentrations of antibody IgG.

### DETAILED DESCRIPTION

Before describing several exemplary embodiments of the invention, it is to be understood that the invention is not limited to the details of construction or method steps set forth in the following description. The invention is capable of other embodiments and of being practiced or being carried out in various ways.

The term “target compound” as used herein refers to an organic compound that can be oxidized, reduced or both. In some embodiments, the organic compound comprises a molecular weight in a range of from 20 Da to 1 MDa. In some embodiments, the organic compound comprises peptides, proteins, nucleic acids, DNA, lipids, carbohydrates, drugs, drug metabolites, synthetic polymers, organic pollutants or combinations thereof. In some embodiments, the organic pollutant comprises per- and polyfluoroalkyl substances (PFAS). In some embodiments, the organic compound has a molecular weight in the range of from 20 Da to 5000 Da such as from 20 Da to 1000 Da, 20 Da to 500 Da, 20 Da to 100 Da, 50 Da to 5000 Da, 50 Da to 1000 Da, 50 Da to 500 Da, 50 Da to 100 Da, 100 Da to 5000 Da, 100 Da to 1000 Da, or 100 Da to 500 Da. In some embodiments, the organic compound comprising a protein has a molecular weight in a range of from 1 kDa to 1 MDa, such as from 5 kDa to 1 MDa, from 50 kDa to 1 MDa, from 100 kDa to 1 MDa, from 500 kDa to 1 MDa, from 1 kDa to 500 kDa, from 5 kDa to 500 kDa, from 50 kDa to 500 kDa, from 100 kDa to 500 kDa, from 1 kDa to 100 kDa, from 5 kDa to 100 kDa, from 50 kDa to 100 kDa, from 1 kDa to 50 kDa, from 5 kDa to 50 kDa, from 1 kDa to 10 kDa, from 5 kDa to 10 kDa, from 1 kDa to 10 kDa or from 1 kDa to 5 kDa. In some embodiments, the organic compound comprising a protein has a molecular weight more than or equal to 100 Da, 500 Da, 1 kDa, 5 kDa, 10 kDa, 50 kDa, 100 kDa, 150 kDa, 200 kDa, 300 kDa, 400 kDa, 500 kDa or 1 MDa. In some embodiments, the target compound comprises insulin protein.

The term “tag” as used herein refers a compound that is electrochemically inactive. In some embodiments, the tag comprises one or more of ferrocene groups and hydroquinone groups. In some embodiments, the target compound further comprises the tag.

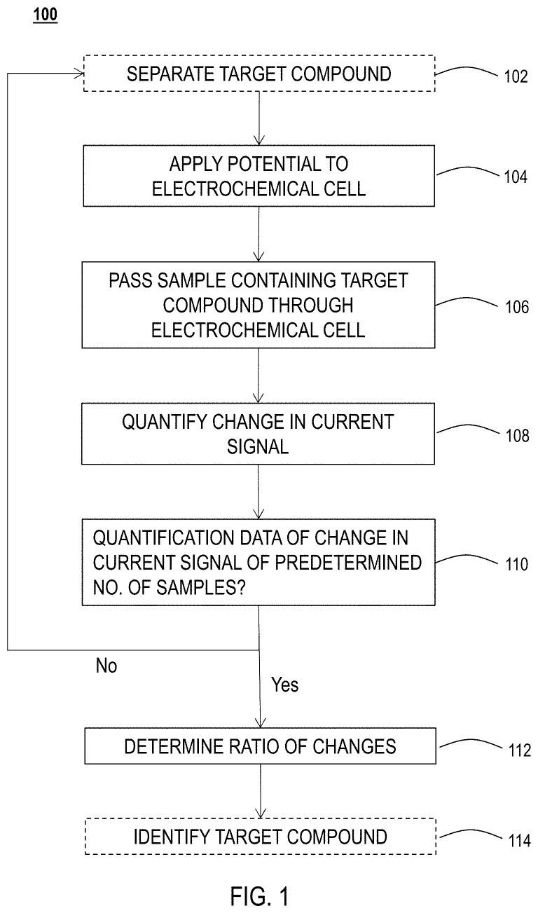

The present disclosure provides methods and systems for determining relative quantities of organic and biological compounds by mass spectrometry mass spectrometry in combination with electrochemistry. FIG. 1 illustrates one or more embodiments of the disclosure directed to a method 100 of determining relative quantity of the target compound in a plurality of samples. In some embodiments, the method 100 advantageously determines relative quantities of intact protein or other large target compound. For relative quantitation, it is not necessary to know exactly how many electrons are transferred to or from the target compound, or how many groups are oxidized/reduced during electrochemical reaction. Therefore, the method 100 can be used even for the target compounds that undergo complicated electrochemical reactions. Accordingly, in some embodiments, the method 100 advantageously determines relative quantities of the target compound. In some embodiments, the method 100 is used to determine relative quantity of the target compound in the plurality of samples, wherein the plurality of samples comprises the target compound in a range of from nanomolar to micromolar.

In some embodiments, the sample comprises a mixture of compounds. At 102, the sample is optionally processed to separate the target compound from the mixture of compounds. In some embodiments, separation of the target compound comprises using one or more or a chromatography device and an electrophoresis device. In some embodiments, the chromatography device comprises Ultra-Performance Liquid Chromatography (UPLC), High-Performance Liquid Chromatography (HPLC) or Nanoscale liquid chromatography (nanoLC).

In some embodiments, one or more compounds of the mixture of compounds are the target compounds. Accordingly, in some embodiments, method 100 is used for determining relative quantities of a plurality of target compounds in a plurality of samples.

At 104, a potential is applied to an electrochemical cell. In some embodiments, the potential comprises oxidative potential, reductive potential or a combination thereof. In some embodiments, the potential is applied in a range of from −2.0 V to +2.0 V. In some embodiments, the potential comprises a direct current potential or a pulsed mode potential.

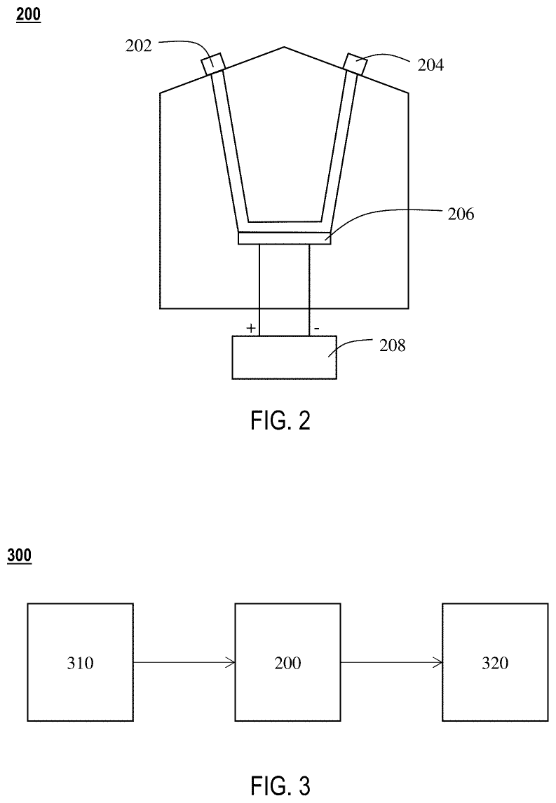

FIG. 2 shows the electrochemical cell 200 according to one or more embodiments of the disclosure. In one or more embodiments, the electrochemical cell comprises an inlet 202, an outlet 204, a potentiostat 208, and one or more electrodes 206. In some embodiments, the potentiostat 208 is configured to measure a current signal detected by one or more electrodes 206 in the presence of the target compound when potential is applied. In some embodiments, the potentiostat 208 is operatively connected with the one or more electrodes 206. In some embodiments, the potentiostat 208 is configured to measure a current signal detected by one or more electrodes 206 in the absence of the target compound when potential is applied.

In some embodiments, the one or more electrodes 206 comprise a reference electrode (RE), a counter electrode (CE), and a working electrode (WE). In some embodiments, the working electrode (WE) is used for oxidating the target compound. In some embodiments, the working electrode (WE) is applied a positive potential in a range of from 0.2 V to 2.0 V for oxidating the target compound. In some embodiments, the reference electrode (RE) comprises HyREF™ electrode, standard hydrogen electrode (SHE), saturated calomel electrode (SCE) or Ag/AgC1 electrode. In some embodiments, the counter electrode (CE) comprises a carbon-loaded PTFE electrode or a stainless-steel block. In some embodiments, the working electrode (WE) comprises a Magic Diamon (boron-doped diamond) disc electrode, glass carbon electrode, platinum electrode or gold electrode. In some embodiments, the one or more electrodes independently comprises a porous electrode, a flat electrode or a modified electrode.

Referring back to FIG. 1, at 106, the sample is passed through the electrochemical cell 200. When a sample analyte passes through the electrochemical cell 200, the target compound is subjected to electrochemical oxidation or reduction, thus causing the rise of a current signal. This signal is proportional to the concentration of the target compound. In some embodiments, the sample containing the target compound is passed through the electrochemical cell 200 at a flowrate in a range of from 0.1 μL/min to 200 μL/min.

At 108, a change in the current signal is determined when the target compound undergoes an electrochemical reaction. According to the Faraday's Law, the electric charge (Q) involved in the oxidation reaction is proportional to the amount (n) of the target compound that has been oxidized: Q=nFz, where n is the mole number of the oxidized target compound, z is the number of electrons transferred per molecule during the electrochemical reaction, F is the Faraday's constant (9.65×10⁴ C/mol).

At decision step 110, if change in current signal is not quantified for a predetermined number of samples, the method moves to step 102 for analysis of second sample. At decision step 110, if change in current signal is quantified for a predetermined number of samples, the method moves to step 112. Under the same oxidation conditions, the target compound in different samples would produce different electric current or charge Q, depending on its concentration. The higher concentration for the target compound, the higher percentage of the target compound is oxidized, thus higher electric current or charge Q will result. Accordingly, in some embodiments, the current or Q is measured to determine relative quantities of the target compound in different samples.

At 112, a ratio of the change in current or Q is calculated to determine relative quantities of the target compound in different samples. In some embodiments, wherein the number of samples are more than two, ratios of the change in current or Q for each sample are calculated relative to each other for determining relative quantities of the target compound in different samples. Change in current or Q due to the electrochemical reaction of the target compound is advantageously more stable, reproducible and avoid ion suppression issue from matrix effect over EIC peak area.

Traditional relative quantitation uses EIC peak area but there are several issues with using EIC peak area of a compound for relative quantitation: (1) EIC signals suffer to ion signal fluctuation and ion suppression from matrix effect of sample during optional upstream or downstream steps of method 100, such as mass spectroscopic analysis and/or liquid chromatographic separation; (2) A relationship between the EIC peak area and concentration of target compound is not linear; and (3) Relative quantitation using EIC is not used for intact proteins because protein produce many ions at different charge states after ionization. Accordingly, one has to consider if one ion peak or all ion peaks will be used for quantitation. Plus, as mentioned above, instrumental signal fluctuation is always an issue, as well as the ion suppression problem from matrix effect.

In one or more embodiments, the method 100 is used to determine relative quantities of peptides and proteins. Accordingly, in some embodiments, a target peptide/protein, if it contains an electrochemically oxidizable amino acid, such as tyrosine, cysteine, tryptophan and/or methionine, is first introduced to an electrochemical cell 200 for electrochemical oxidation. Accordingly, the electric charge (Q) involved in the oxidation reaction is proportional to the amount (n) of the peptide that has been oxidized: Q=nFz, where n is the mole number of the oxidized peptide/protein, z is the number of electrons transferred per molecule during the electrochemical reaction, F is the Faraday's constant (9.65×10⁴ C/mol).

Under the same oxidation conditions, the peptide/protein in different samples would produce different electric current or charge Q, depending on its concentration. The higher concentration for the peptide/protein, the higher percentage of the peptide/protein is oxidized, thus higher electric current or charge Q will result. By measuring the current or Q, the present inventors could conduct relative quantitation for peptide/protein in different samples.

At 114, optionally, the target compound is identified. In some embodiments, the target compound is identified based upon molecular weight and structural information of the target compound. In some embodiments, the target compound is identified by a method comprising one or more of a mass spectrometry, a UV-Vis spectroscopy, and a fluorescence spectroscopy. Accordingly, in some embodiments, the electrochemical cell 200 is operatively connected to a mass spectrometer, a UV-Vis spectrometer or a fluorescence spectrometer via the outlet 204. In some embodiments, the target compound is identified by the mass spectrometry. In some embodiments, the mass spectrometry comprises ionizing the target compound. In some embodiments, ionizing the target compound comprises electrospray ionization, laser ionization, plasma ionization, high energy particle ionization or combinations thereof. In some embodiments, ionizing the target compound comprises electrospray ionization. In some embodiments, the target compound is maintained under a potential in a range of from −5.0 kV to +5.0 kV when processed through electrospray ionization. In some embodiments, the sample containing the target compound is processed through the electrospray ionization at a flowrate in a range of from 0.1 μL/min to 1000 μL/min.

FIG. 3 is a schematic of one embodiment of a relative quantification system 300. The quantification system comprises the electrochemical cell 200 is operatively connected to a detection device 320 (e.g. a mass spectrometer (MS)) and a separating device 310. In some embodiments, the separating device 310 comprises a liquid chromatography (LC) device and/or an electrophoresis device. The separating device 310 separates the target compound from other components in the sample prior to infusing into the electrochemical cell 200. In some embodiments, direct infusion may be employed for a sample that is pure (i.e. only contains the target compound(s)) or that is a simple mixture composed of only a small number of constituents. In some embodiments, the detection device comprises a mass spectrometer (MS). In some embodiments, the electrochemical cell could include a reference electrode (RE), a counter electrode (CE), and a working electrode (WE).

When a sample analyte passes through the electrochemical cell, the sample analyte is subjected to electrochemical oxidation and/or reduction, thus causing the rise of an electrochemical current signal. This signal is proportional to the concentration of the analyte. In some embodiments, the target compound is subject to electrospray ionization treatment followed by MS detection for identification. Thus, by this approach, identification and relative quantitation of the target compound in samples can be conducted simultaneously.

One embodiment of the method 100 is for relative quantitation of peptides and proteins, wherein the target peptide/protein, if it contains an electrochemically oxidizable amino acid like tyrosine (or cysteine, tryptophan and methionine), is first introduced to an electrochemical cell 200 for electrochemical oxidation and followed by mass spectrometry detection. Accordingly, relative quantitation for peptide/protein in different samples and mass spectrometric detection can be conducted simultaneously.

According to the Faraday's Law, the electric charge (Q) involved in the oxidation reaction is proportional to the amount (n) of the peptide that has been oxidized: Q=nFz, where n is the mole number of the oxidized peptide/protein, z is the number of electrons transferred per molecule during the redox reaction, F is the Faraday's constant (9.65×10⁴ C/mol). Under the same oxidation conditions, the peptide/protein in different samples would produce different electric current or charge Q, depending on its concentration. The higher concentration for the peptide/protein, the higher percentage of the peptide/protein is oxidized, thus higher electric current or charge Q will result. By measuring the current or Q, the present inventors could conduct relative quantitation for peptide/protein in different samples. Furthermore, simultaneous mass spectrometric detection can be used for peptide/protein identification.

In some embodiments, the liquid chromatography device is from (Waters, Milford, Mass.). In some embodiments, the electrochemical cell 200 is from (BASi, West Lafayette, Ind.). In some embodiments, the mass spectrometer comprises a high resolution Orbitrap QE mass spectrometer (Thermo Scientific, San Jose, Calif.). In some embodiments, the electrochemical cell 200 comprises a thin-layer electrochemical flow cell. In some embodiments, the thin-layer electrochemical flow cell comprises a glass carbon as the working electrode, an Ag/AgCl electrode as the reference electrode, and a stainless-steel block as the counter electrode. In some embodiments, the potentiostat 208 is used to apply potential to the thin-layer electrochemical cell so that it results in oxidization or reduction of species in the thin-layer electrochemical cell. In some embodiments, the target peptide can be separated out from the liquid chromatography device first, then electrochemically oxidized by the electrochemical cell 200, and finally the peptide can be identified by mass spectrometry.

The materials and the methods of the present disclosure used in one embodiment will be described below. While the embodiment discusses the use of specific compounds and materials, it is understood that the present disclosure could employ other suitable compounds or materials. Similar quantities or measurements may be substituted without altering the method embodied below.

### EXAMPLE 1

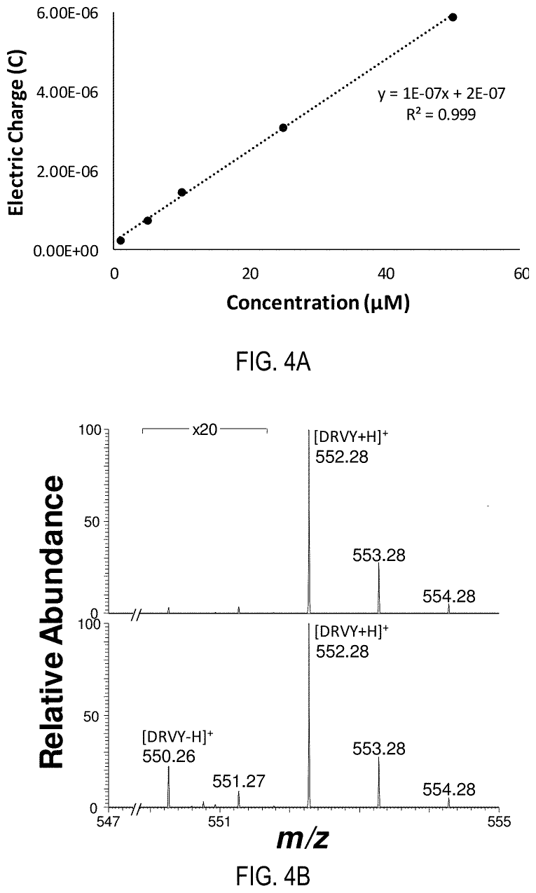

By using the setup according to FIG. 3, a setup of a coupling liquid chromatography device (Waters, Milford, Mass.), an electrochemical cell (BASi, West Lafayette, Ind.), and a high resolution Orbitrap QE mass spectrometer (Thermo Scientific, San Jose, Calif.) was made to test a model peptide DRVY at different concentrations (0.1 μM, 1 μM, 10 μM and 50 μM). While the peptide DRVY at different concentrations was oxidized in the electrochemical cell, the resulting electric current was integrated and found to be proportional to the peptide concentration. Referring to FIG. 4A, the plot of the integrated current with time (i.e., the electricity Q involved in the DRVY oxidation) vs. the sample concentration is linear (R²=0.999). Meanwhile the DRVY and its oxidization products were clearly detected by high resolution MS, as shown in FIG. 4B.

### EXAMPLE 2

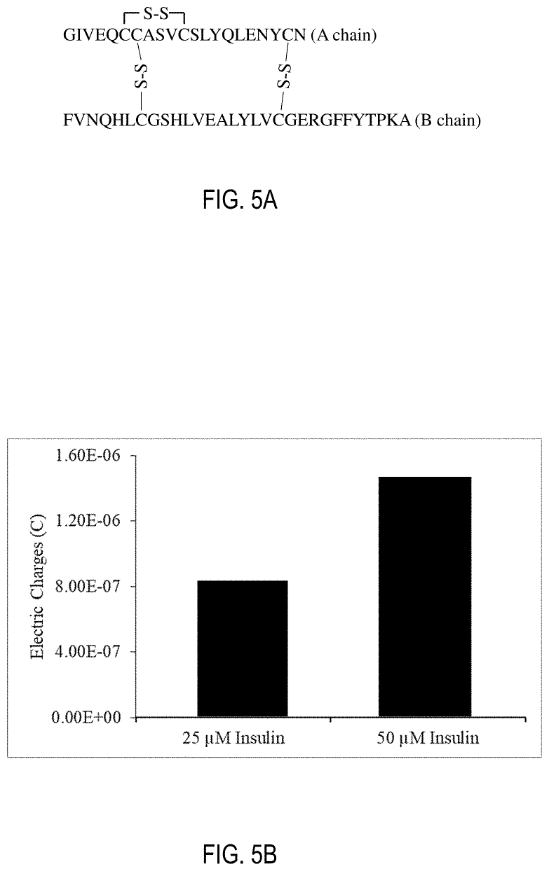

Protein insulin was examined using the method 100. FIG. 5A shows the protein sequence. FIG. 5B shows the integrated oxidation charge Q from the oxidation of two insulin samples at 25 μM and 50 μM concentrations. As shown in FIG. 5B, the electric charge Q from insulin oxidation measured to be 8.10E-7 C and 1.57E-6 C, respectively, for the two samples. Thus, the ratio of Qs from the two samples is 1.57E-6/8.10E-7=1.938, which is fairly close to the concentration ratio of 1:2.

### EXAMPLE 3

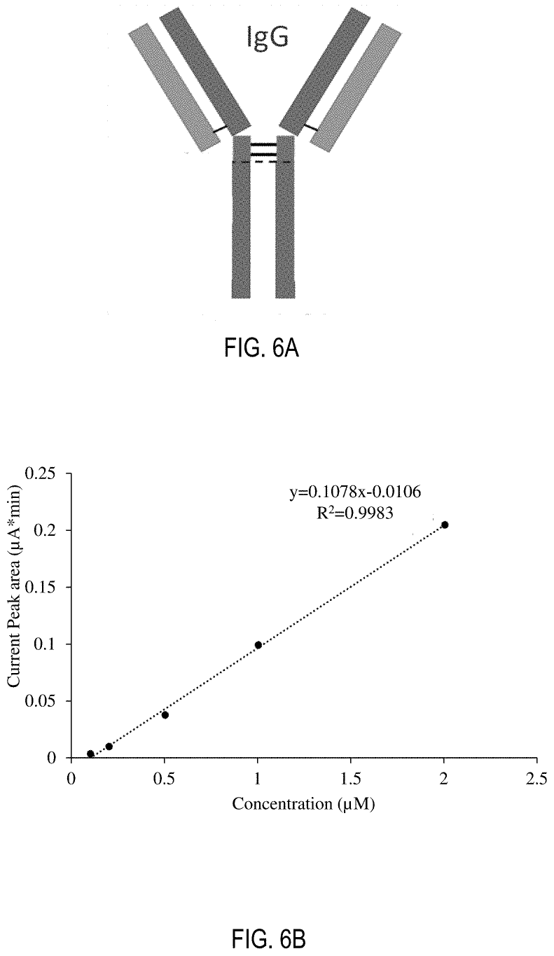

A large NIST antibody protein IgG was examined using the method 100. The working electrode (WE) used for IgG was a home-designed boron-doped diamond (BDD) planar disc electrode (6 mm ID) and a pulsed potential sequence was used to trigger the protein oxidation and electrode cleaning. FIG. 6A shows the protein structure which has two heavy and light chains that are connected with disulfide bonds. FIG. 6B shows the plot of integrated electric current peak area (i.e., Q) vs. different concentrations of IgG from 0.1-2 μM. As shown in FIG. 6B, the plot linearity is excellent with R²=0.9983, suggesting that Q from IgG electrochemical oxidation does proportionally correlate to the IgG concentration.

These results show the feasibility of using an oxidation electric current or electricity to relatively quantify peptide/protein concentrations. Higher electric current/Q corresponds to higher sample concentration. The experimental protocol is simple and could be used to measure many samples quickly. The major strength is that no standards or calibration curves for quantitation are needed. Therefore, the present method is fast and cost effective. Furthermore, it is implementable in traditional LC/MS platform, which is ubiquitous in both academy and industry. As a liquid chromatography device is used in one embodiment of the method 100, target compounds in a complex sample can be separated and then quantified. Meanwhile, MS can be used for analyte identification. The method 100 could make a high impact in proteomics, metabolomics fields and find many useful analytical and bioanalytical applications in the future.

In the liquid chromatography/electrochemical cell 200/mass spectrometry setup, the liquid chromatography (LC) device can be any suitable liquid chromatography instrument like UPLC, HPLC or nanoLC. Alternatively, an electrophoresis device can be used to replace the LC device for separation. The mass spectrometry device can be any suitable mass spectrometer instrument with an atmospheric pressure interface.

The ionization method is not limited to electrospray ionization (ESI) but also can be any suitable ionization method based on laser, high energy particles, or plasmas. The electrochemical cell can be any suitable electrochemical cell employing a porous electrode, a flat electrode, or a modified electrode. In some embodiments, the electrochemical cell 200 further comprises a faraday cage, a current amplifier, or a combination thereof to improve the sensitivity of method 100.

The method 100 can be automated and can perform high throughput analysis for many samples quickly. The present system could include subsystems and components to measure and control process variables, as required for effective performance. The present system could employ sensors or other condition detection and control subsystems or components that might be required to process at a particular rate or at a particular scale. For example, a sensor could monitor and record an electrochemical current during an oxidation or reduction process in the electrochemical cell 200. The present system could include a controller in communication with a sensor. The controller could receive at least one process parameter, process the at least one process parameter, and adjust operation of the system based upon processing of the at least one process parameter.

### REFERENCES

(1) Lindemann, C.; Thomanek, N.; Hundt, F.; Lerari, T.; Meyer, H. E.; Wolters, D.; Marcus, K. Strategies in Relative and Absolute Quantitative Mass Spectrometry Based Proteomics. Biol. Chem. 2017, 398 (5-6), 687-699.
(2) Ong, S.-E.; Blagoev, B.; Kratchmarova, I.; Kristensen, D. B.; Steen, H.; Pandey, A.; Mann, M. Stable Isotope Labeling by Amino Acids in Cell Culture, SILAC, as a Simple and Accurate Approach to Expression Proteomics. Mol. Cell. Proteomics 2002, 1 (5), 376-386.
(3) Andrew Thompson; Jurgen Schafer; Karsten Kuhn; Stefan Kienle; Josef Schwarz; Gunter Schmidt; Thomas Neumann, and; Christian Hamon. Tandem Mass Tags: A Novel Quantification Strategy for Comparative Analysis of Complex Protein Mixtures by MS/MS. Anal Chem 2003, 75 (8), 1895-1904.
(4) Zhang, L.; Elias, J. E. Relative Protein Quantification Using Tandem Mass Tag Mass Spectrometry. In Methods in molecular biology (Clifton, N. J.); 2017; Vol. 1550, pp 185-198.
(5) Gygi, S. P.; Rist, B.; Gerber, S. A.; Turecek, F.; Gelb, M. H.; Aebersold, R. Quantitative Analysis of Complex Protein Mixtures Using Isotope-Coded Affinity Tags. Nat. Biotechnol. 1999, 17 (10), 994-999.
(6) Wiese, S.; Reidegeld, K. A.; Meyer, H. E.; Warscheid, B. Protein Labeling by ITRAQ: A New Tool for Quantitative Mass Spectrometry in Proteome Research. Proteomics 2007, 7 (3), 340-350.
(7) Xu, C.; Zheng, Q.; Zhao, P.; Paterson, J.; Chen, H. A New Quantification Method Using Electrochemical Mass Spectrometry. J. Am. Soc. Mass Spectrom. 2019, 30 (4), 685-693.
(8) Zhao, P.; Guo, Y.; Dewald, H. D.; Chen, H. Improvements for Absolute Quantitation Using Electrochemical Mass Spectrometry. Int. J. Mass Spectrom. 2019, 443, 41-45.
(9) Zhao, P.; Zare, R. N.; Chen, H. Absolute Quantitation of Oxidizable Peptides by Coulometric Mass Spectrometry. J. Am. Soc. Mass Spectrom. 2019, 30, 2398-2407.
(10) CHEN, H. A New Method and Device for Chemical Quantification Using Electrochemical Mass Spectrometry Without the Use of Standard Target Compounds. WO/2018/081228, May 3, 2018.
(11) Johnson, D. C., LaCourse, W. R. Liquid chromatography with pulsed electrochemical detection at gold and platinum electrodes, Anal. Chem., 1990, 62, 589A-597A
(12) Sadik, O. A.; Wallace, G. G., Pulse damperometric detection of proteins using antibody containing conducting polymers, Anal. Chim. Acta. 1993, 279, 209.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

### Embodiments

Embodiment (a). A method for determining relative quantity of a target compound in a plurality samples, the method comprising: applying a first potential to an electrochemical cell; passing a first sample containing the target compound through the electrochemical cell; quantifying a first change in an electrochemical current signal flowing through the electrochemical cell; applying a second potential to the electrochemical cell; passing a second sample containing the target compound through the electrochemical cell; quantifying a second change in the electrochemical current signal flowing through the electrochemical cell; and determining a ratio of the first change in the electrochemical current signal and the second change in the electrochemical current signal.

Embodiment (b). The method of embodiment (a), wherein the potential comprises oxidative potential, reductive potential or a combination thereof.

Embodiment (c). The method of embodiment (a) or (b), wherein the potential comprises direct current potential and a pulsed mode potential.

Embodiment (d). The method of any one of embodiments (a)-(c), further comprising: applying a third potential to the electrochemical cell; passing a third sample containing the target compound through the electrochemical cell; quantifying a third change in the electrochemical current signal flowing through the electrochemical cell; and determining a ratio of the first change in the electrochemical current signal and the third change in the electrochemical current signal, and/or the second change in the electrochemical current signal and the third change in the electrochemical current signal.

Embodiment (e). The method of any one of embodiments (a)-(d), further comprising identifying the target compound.

Embodiment (f). The method of embodiment (e), wherein the target compound is identified based upon molecular weight and structural information of the target compound.

Embodiment (g). The method of embodiment (e) or (f), wherein the target compound is identified by a method comprising one or more of a mass spectrometry, a UV-Vis spectroscopy, and a fluorescence spectroscopy.

Embodiment (h). The method of any one of embodiments (e) to (g), wherein the target compound is identified by mass spectrometry.

Embodiment (i). The method of embodiment (h), wherein the mass spectrometry comprises ionizing the target compound.

Embodiment (j). The method of embodiment (i), wherein ionizing the target compound comprises electrospray ionization, laser ionization, plasma ionization, high energy particle ionization or combinations thereof.

Embodiment (k). The method of any one of embodiments (a) to (j), further comprising separating the target compound from a mixture in the first sample and/or the second sample.

Embodiment (l). The method of embodiment (k), wherein separating the target compound comprises using one or more or a chromatography device and an electrophoresis device.

Embodiment (m). The method of embodiment (l), wherein the chromatography device comprises Ultra-Performance Liquid Chromatography (UPLC), High-Performance Liquid Chromatography (HPLC) or Nanoscale liquid chromatography (nanoLC).

Embodiment (n). The method of any one of embodiments (a) to (m), wherein the target compound comprises peptides, proteins, nucleic acids, lipids, carbohydrates, drugs, drug metabolites, synthetic polymers, organic pollutants or combinations thereof.

Embodiment (o). The method of embodiment (n), wherein the organic pollutant comprises per- and polyfluoroalkyl substances (PFAS).

Embodiment (p). The method of any one of embodiments (a) to (o), wherein the electrochemical cell comprises an inlet, an outlet, an electrode and a controller, the controller is configured to measure a current signal of the target compound.

Embodiment (q). The method of embodiment (p), wherein the electrode comprises a porous electrode, a flat electrode, or a modified electrode.

Embodiment (r). The method of embodiment (p) or (q), wherein a mass spectrometer is operatively connected to the outlet for detecting the target compound.

Embodiment (s). The method of any one of embodiments (p) to (r), wherein a liquid chromatography or an electrophoresis device is operatively connected to the inlet for separating the target compound from a mixture containing the target compound in the first sample and/or the second sample.

Embodiment (t). The method of any one of embodiments (a) to (s), wherein the target compound has a molecular weight in a range of from 20 Da to 1 MDa.

What is claimed is:

1. A method for determining relative quantity of a target compound in a plurality of samples, the method comprising:
applying a first potential to an electrochemical cell;
passing a first sample containing the target compound through the electrochemical cell;
quantifying a first change in an electrochemical current signal flowing through the electrochemical cell;
applying a second potential to the electrochemical cell;
passing a second sample containing the target compound through the electrochemical cell;
quantifying a second change in the electrochemical current signal flowing through the electrochemical cell; and
determining a ratio of the first change in the electrochemical current signal and the second change in the electrochemical current signal.

2. The method of claim 1, wherein the potential comprises oxidative potential, reductive potential or a combination thereof.

3. The method of claim 1, wherein the potential comprises direct current potential and a pulsed mode potential.

4. The method of claim 1 further comprising:
applying a third potential to the electrochemical cell;
passing a third sample containing the target compound through the electrochemical cell;
quantifying a third change in the electrochemical current signal flowing through the electrochemical cell; and
determining a ratio of (i) the first change in the electrochemical current signal and the third change in the electrochemical current signal, and/or (ii) the second change in the electrochemical current signal and the third change in the electrochemical current signal.

5. The method of claim 1, further comprising identifying the target compound.

6. The method of claim 5, wherein the target compound is identified based upon molecular weight and structural information of the target compound.

7. The method of claim 5, wherein the target compound is identified by a method comprising one or more of a mass spectrometry, a UV-VIS spectroscopy, and a fluorescence spectroscopy.

8. The method of claim 5, wherein the target compound is identified by mass spectrometry.

9. The method of claim 8, wherein the mass spectrometry comprises ionizing the target compound.

10. The method of claim 9, wherein ionizing the target compound comprises electrospray ionization, laser ionization, plasma ionization, high energy particle ionization or combinations thereof.

11. The method of claim 1, further comprising separating the target compound from a mixture in the first sample and/or the second sample.

12. The method of claim 11, wherein separating the target compound comprises using one or more or a chromatography device and an electrophoresis device.

13. The method of claim 12, wherein the chromatography device comprises Ultra-Performance Liquid Chromatography (UPLC), High-Performance Liquid Chromatography (HPLC) or Nanoscale liquid chromatography (nanoLC).

14. The method of claim 1, wherein the target compound has a molecular weight in a range of from 20 Da to 1 MDa.

15. The method of claim 1, wherein the target compound comprises peptides, proteins, nucleic acids, lipids, carbohydrates, drugs, drug metabolites, synthetic polymers, organic pollutants or combinations thereof.

16. The method of claim 15, wherein the organic pollutant comprises per- and polyfluoroalkyl substances (PFAS).

17. The method of claim 1, wherein the electrochemical cell comprises an inlet, an outlet, an electrode and a controller, the controller is configured to measure a current signal of the target compound.

18. The method of claim 17, wherein the electrode comprises a porous electrode, a flat electrode, or a modified electrode.

19. The method of claim 17, wherein a mass spectrometer is operatively connected to the outlet for detecting the target compound.

20. The method of claim 17, wherein a liquid chromatography or an electrophoresis device is operatively connected to the inlet for separating the target compound from a mixture containing the target compound in the first sample and/or the second sample.
